(19) United States
(12) Patent Application Publication
(10) Pub. No.: US 2025/0073267 A1
(43) Pub. Date: Mar. 6, 2025

(54) CYTOMEGALOVIRUS VECTORS AND METHODS OF USE

(71) Applicant: Mayo Foundation for Medical Education and Research, Rochester, MN (US)

(72) Inventors: Haifei Jiang, Rochester, MN (US); Stephen James Russell, Rochester, MN (US)

(21) Appl. No.: 18/726,619

(22) PCT Filed: Jan. 4, 2023
(86) PCT No.: PCT/US2023/060081

§ 371 Date: Jul. 3, 2024

Related U.S. Application Data
(60) Provisional application No. 63/297,339, filed on Jan. 7, 2022.

(51) Int. Cl. A61K 35/17; A61K 39/00; C07K 14/005; C07K 14/725; C12N 15/86

(52) U.S. Cl. CPC A61K 35/17; A61K 40/11; A61K 40/31; A61K 40/4211; C07K 14/005; C07K 14/7051; C12N 15/86; C12N 2710/16143; C12N 2710/16144; C12N 2740/13022; C12N 2740/16043; C12N 2760/20222; C12N 2800/30

(57) ABSTRACT

This document relates to methods and materials involved in treating a mammal (e.g., a human) having cancer and/or an infectious disease. For example, this document provides recombinant human cytomegalovirus (hCMV) vectors that include (e.g., are designed to include) nucleic acid encoding a viral gene transfer vector genome (e.g., a heterologous viral gene transfer vector genome) and one or more nucleic acids encoding a packaging polypeptide such that a cell of a mammal that is infected with the hCMV vector can produce and release the viral vector (e.g., an infectious lentiviral vector) which can then infect cells (e.g., immune cells) in vivo and can, optionally, drive expression of an exogenous polypeptide (e.g., a therapeutic polypeptide or an antigen receptor such as a chimeric antigen receptor (CAR)) in the infected immune cells within a mammal (e.g., a human) to induce an immune response within the mammal are provided.

### CROSS-REFERENCE TO RELATED APPLICATIONS

This application claims the benefit of U.S. Patent Application Ser. No. 63/297,339, filed on Jan. 7, 2022. The disclosure of the prior application is considered part of (and is incorporated by reference in) the disclosure of this application.

### SEQUENCE LISTING

This application contains a Sequence Listing that has been submitted electronically as an XML file named “07039-2095WO1_SL.xml.” The XML file, created on Dec. 12, 2022, is 10000 bytes in size. The material in the XML file is hereby incorporated by reference in its entirety.

### TECHNICAL FIELD

This document relates to methods and materials involved in treating a mammal (e.g., a human) having cancer and/or an infectious disease. For example, this document provides recombinant human cytomegalovirus (hCMV) vectors that include (e.g., are designed to include) nucleic acid encoding a viral gene transfer vector genome (e.g., a heterologous viral gene transfer vector genome) and one or more nucleic acids encoding a packaging polypeptide such that a cell of a mammal that is infected with the hCMV vector can produce and release the viral vector (e.g., an infectious lentiviral vector which can then infect cells (e.g., immune cells) in vivo and/or can be recognized and internalized by an antigen presenting cell (APC)) and can, optionally, drive expression of an exogenous polypeptide (e.g., a therapeutic polypeptide or an antigen receptor such as a chimeric antigen receptor (CAR)) in the infected immune cells within a mammal (e.g., a human) to induce an immune response within the mammal. In some cases, recombinant hCMV vectors provided herein can be administered to a mammal having cancer and/or an infectious disease to induce an immune response against the cancer and/or a pathogen causing the infectious disease within the mammal (e.g., to treat the mammal).

### BACKGROUND INFORMATION

Viral vectors are reliable tools that can transfer foreign genetic materials or genome-manipulating components into target cells (Warnock et al., Methods Mol. Biol., 737:1-25 (2011)). For example, lentivirus vectors can integrate exogenous genes into a host genome, while adeno-associated virus (AAV) vectors can be produced at high titer and present feasible in vivo infection efficiency (Maes et al., Neurosci. Lett., 707:134310 (2019)).

### SUMMARY

Current protocols for production of genetically modified T cells expressing a CAR (CAR T cells) include isolation of autogenous T cells from a patient, activation and propagation of isolated T cells with cytokines, ex vivo transduction of the T cells with a lentivirus designed to express a CAR, thereby forming CAR T cells, and transfusion of the CAR T cells back into the patient (Tyagarajan et al., Mol. Ther. Methods Clin. Dev., 16:136-144 (2019)). This disclosure is based, at least in part, on the design of a recombinant hCMV that can be administered to a patient to achieve immune cell editing (e.g., CAR T cell generation) in vivo in a single step.

This document relates to methods and materials involved in treating a mammal (e.g., a human) having cancer and/or an infectious disease. For example, this document provides recombinant hCMV vectors that include (e.g., are designed to include) nucleic acid encoding a viral vector genome (e.g., a heterologous viral gene transfer vector genome) and one or more nucleic acids encoding a packaging polypeptide, and are capable of infecting cells within a mammal. In some cases, the hCMV vector-infected cells can produce and release a viral vector (e.g., a heterologous viral gene transfer vector such as a lentiviral vector) that can infect other cells (e.g., immune cells such as T cells) in vivo and can, optionally, drive expression of an exogenous polypeptide (e.g., a therapeutic polypeptide or an antigen receptor such as a CAR) in the viral vector-infected cells within a mammal (e.g., a human) to induce an immune response within the mammal. In some cases, the hCMV vector-infected cells can produce and release a viral vector (e.g., a heterologous viral gene transfer vector such as a lentiviral vector) derived from a virus that can cause an infectious disease and can be recognized as a pathogen by an APC in vivo such that the viral vector is internalized the by APC and one or more polypeptides from the viral vector are presented as antigens to one or more immune cells (e.g., T cells) to induce an immune response within the mammal. This document also provides methods and materials for making and using such recombinant hCMV vectors. In some cases, recombinant hCMV vectors provided herein can be administered to a mammal having cancer to induce an immune response against the cancer within the mammal (e.g., to treat the mammal). In some cases, recombinant hCMV vectors provided herein can be administered to a mammal having an infectious disease to induce an immune response against a pathogen causing the infectious disease within the mammal (e.g., to treat the mammal).

As described herein, administering a population of recombinant hCMVs designed to infect cells within a mammal to produce hCMV vector-infected cells within the mammal that in turn produce and release a viral vector (e.g., a heterologous viral gene transfer vector such as a lentiviral vector) that can infect other cells (e.g., T cells) in vivo can result in the in vivo editing of one or more immune cell functions of the viral vector-infected cells (e.g., the lentiviral vector-infected T cells). For example, a recombinant hCMV vector provided herein can infect a cell within a mammal (e.g., a human) and can produce one or more endogenous hCMV polypeptides to recruit one or more immune cells to the infected cell (e.g., a hCMV vector-infected fibroblast, hCMV vector-infected endothelial cell, hCMV vector-infected monocyte, or hCMV vector-infected glial cell) such that a viral vector produced by and released from the hCMV vector-infected cell can infect a recruited immune cell. In some cases, a viral vector produced by and released from a hCMV vector-infected cell can include nucleic acid encoding an exogenous polypeptide (e.g., a therapeutic polypeptide and/or an antigen receptor such as a CAR) such that the when the viral vector infects a recruited immune cell, the viral vector-infected recruited immune cell can express the exogenous polypeptide. For example, a recombinant hCMV vector provided herein can be designed to produce a viral vector (e.g., a heterologous viral gene transfer vector such as a lentiviral vector) including nucleic acid encoding a therapeutic polypeptide such that the viral vector can infect a recruited immune cell (e.g., a T cell) and the infected recruited immune cell can express the therapeutic polypeptide. For example, a recombinant hCMV vector provided herein can be designed to produce a viral vector (e.g., a heterologous viral gene transfer vector such as a lentiviral vector) including nucleic acid encoding a CAR such that the viral vector can infect a recruited immune cell (e.g., a T cell) and the infected recruited immune cell can express the CAR resulting in the in vivo generation of a CAR immune cell (e.g., a CAR⁺ T cell). Also as demonstrated herein, recombinant hCMVs provided herein can be further designed to have altered tropism. For example, a recombinant hCMV can be designed to include a nucleic acid sequence encoding a functional UL131 polypeptide (e.g., a UL131 nucleic acid sequence from a Merlin strain of hCMV) that can allow the hCMV to infect cells that are not fibroblasts such as epithelial cells, endothelial cells, monocytes, and glial cells.

The ability to induce an immune response within a mammal, and, optionally, to generate CAR T cells within a mammal as described herein provides a unique opportunity to use immunotherapy to target (e.g., to locate and destroy) particular cells (e.g., disease cells such as cancer cells and infected cells such as virus infected cells).

In general, one aspect of this document features recombinant hCMV vectors capable of infecting a cell of a mammal. The hCMV vector can include heterologous nucleic acid encoding (i) a heterologous viral gene transfer vector genome and (ii) one or more helper polypeptides for amplifying and packaging the heterologous viral gene transfer vector genome into infectious vector particles, where the cell infected with the hCMV vector produces and releases the infectious vector particles comprising the gene transfer vector genome. The hCMV vector can be replication-competent in the cell. The hCMV vector can lack nucleic acid encoding a UL138 polypeptide, a UL144 polypeptide, a UL146 polypeptide, a UL147 polypeptide, or a miR-UL148D. The hCMV vector can lack a UL138 polypeptide, a UL144 polypeptide, a UL146 polypeptide, a UL147 polypeptide, or a miR-UL148D. The hCMV vector can be derived from an hCMV AD169 strain or an hCMV Merlin strain. The hCMV vector can lack nucleic acid encoding a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The hCMV vector can lack a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The cell can be a fibroblast, an epithelial cell, an endothelial cell, a monocyte, or a glial cell. The viral gene transfer vector genome can be a lentiviral vector genome, a retroviral vector genome, an AAV vector genome, a picornavirus vector genome, a rhabdovirus vector genome, or a coronavirus vector genome. The viral gene transfer vector genome can include a nucleic acid sequence encoding a therapeutic polypeptide or an antigen receptor polypeptide, where the infectious vector particles comprising the gene transfer vector genome can infect an immune cell within the mammal, and where the infected immune cell can direct expression of the therapeutic polypeptide or the antigen receptor polypeptide. In some cases, the viral gene transfer vector genome can include a nucleic acid sequence encoding the therapeutic polypeptide. The therapeutic polypeptide can be a chemokine, a cytokine, an anti-PD1 antibody, an anti-PDL-1 antibody, an anti-CTLA-4 antibody, or a CD47 polypeptide. In some cases, the viral gene transfer vector genome can include a nucleic acid sequence encoding the antigen receptor polypeptide. The antigen receptor polypeptide can be a CAR polypeptide (e.g., an anti-CD19 CAR). The immune cell can be a T cell, a natural killer (NK) cell, or a natural killer T (NKT) cell. The viral gene transfer vector can be replication-defective in the infected immune cell. The viral gene transfer vector genome can include one or more packaging elements selected from the group consisting of a 5′ long terminal repeat (LTR), a 3′ LTR, a psi (Ψ) element, a Rev response element (RRE), and a central polypurine tract/central termination sequence (cPPT/CTS). The nucleic acid encoding one or more helper polypeptides can include nucleic acid encoding an envelope polypeptide selected from the group consisting of a vesicular stomatitis virus G (VSV G) polypeptide and a murine leukemia virus (MLV) 4070A polypeptide. The released viral gene transfer vector can include the envelope polypeptide.

In another aspect, this document features methods for treating a mammal having cancer. The methods can include, or consist essentially of, administering a recombinant hCMV vector to a mammal having cancer, where the hCMV vector is capable of infecting a cell of the mammal, where the hCMV includes heterologous nucleic acid encoding (i) a heterologous viral gene transfer vector genome including a nucleic acid sequence encoding an antigen receptor polypeptide targeting a cancer antigen of the cancer and (ii) one or more helper polypeptides for amplifying and packaging the heterologous viral gene transfer vector genome into infectious vector particles, where the cell infected with the hCMV produces and releases the infectious vector particles comprising the gene transfer vector genome, and where the released infectious vector particles are capable of infecting an immune cell within the mammal and directing expression of the antigen receptor polypeptide by the infected immune cell, and where the infected immune cell expressing the antigen receptor reduces the number of cancer cells within the mammal. The mammal can be a human. The cancer can be a brain cancer, a B cell lymphoma, an ALL, a CLL, a neuroblastoma, a breast cancer, or a lung cancer. The cancer antigen can be cluster of differentiation 19 (CD19), alphafetoprotein (AFP), carcinoembryonic antigen (CEA), CA-125, mucin 1 (MUC-1), epithelial tumor antigen (ETA), melanoma-associated antigen (MAGE), human epidermal growth factor receptor 2 (HER2), biotin, EGFRvIII, CD171, or mesothelin. The hCMV vector can be replication-competent in the cell. The hCMV vector can lack nucleic acid encoding a UL138 polypeptide, a UL144 polypeptide, a UL146 polypeptide, a UL147 polypeptide, or a miR-UL148D. The hCMV vector can lack a UL138 polypeptide, a UL144 polypeptide, a UL146 polypeptide, a UL147 polypeptide, or a miR-UL148D. The hCMV vector can be derived from an hCMV AD169 strain or an hCMV Merlin strain. The hCMV vector can lack nucleic acid encoding a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The hCMV vector can lack a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The cell can be a fibroblast, an epithelial cell, an endothelial cell, a monocyte, or a glial cell. The viral gene transfer vector genome can be a lentiviral vector genome, a retroviral vector genome, an AAV vector genome, a picornavirus vector genome, a rhabdovirus vector genome, or a coronavirus vector genome. The viral gene transfer vector can include nucleic acid encoding one or more packaging elements selected from the group consisting s 5′ LTR, a 3′ LTR, a psi (Ψ) element, a RRE, and a cPPT/CTS. The nucleic acid encoding one or more helper polypeptides can include nucleic acid encoding an envelope polypeptide selected from the group consisting of a VSV G protein or a MLV 4070A protein. The released viral gene transfer vector can include the envelope polypeptide.

In another aspect, this document features methods for treating a mammal having an infectious disease. The methods can include, or consist essentially of, administering a recombinant hCMV vector to a mammal having an infectious disease, where the hCMV vector is capable of infecting a cell of the mammal, where the hCMV vector includes heterologous nucleic acid encoding (i) a heterologous viral gene transfer vector genome derived from a virus causing the infectious disease and (ii) one or more helper polypeptides for amplifying and packaging the heterologous viral gene transfer vector genome into infectious vector particles, where the cell infected with the hCMV vector produces and releases the infectious vector particles comprising the gene transfer vector genome, and where the infectious vector particles are recognized by an APC within the mammal such that the APC presents an antigen from the viral gene transfer vector to a T cell within the mammal, and where the T cell recognizes and destroys the virus causing the infectious disease within the mammal. The mammal can be a human. The infectious disease can be viral pneumonia, acquired immune deficiency syndrome, or viral hepatitis. The hCMV vector can be replication-competent in the cell. The hCMV vector can lack nucleic acid encoding a UL138 polypeptide, a UL144 polypeptide, a UL146 polypeptide, a UL147 polypeptide, or a miR-UL148D. The hCMV vector can lack a UL138 polypeptide, a UL144 polypeptide, a UL146 polypeptide, a UL147 polypeptide, or a miR-UL148D. The hCMV vector can be derived from an hCMV AD169 strain or an hCMV Merlin strain. The hCMV vector can lack nucleic acid encoding a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The hCMV vector can lack a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide. The cell can be a fibroblast, an epithelial cell, an endothelial cell, a monocyte, or a glial cell. The viral gene transfer vector genome can be a lentiviral vector genome, a retroviral vector genome, an AAV vector genome, a picornavirus vector genome, a rhabdovirus vector genome, or a coronavirus vector genome. The viral gene transfer vector genome can include nucleic acid encoding one or more packaging elements selected from the group consisting of 5′ LTR, a 3′ LTR, a psi (Ψ) element, a RRE, and a cPPT/CTS. The nucleic acid encoding one or more helper polypeptides can include nucleic acid encoding an envelope polypeptide selected from the group consisting of a VSV G protein or a MLV 4070A protein. The released viral gene transfer vector can include the envelope polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Construction of an exemplary hCMV-BAC. The BAC vector containing left homology arm (hCMV unique short (US) US27-28 sequence) and right homology arm (hCMV US29-30 sequence) was linearized and co-transfected with AD169 or Merlin linear genome into MRC-5 cells to generate the recombinant hCMV-BAC viruses. Green plaques of the recombinant virus were purified and then circular viral DNA was isolated from the hCMV-BAC infected cells. The circular viral BAC DNA was transformed into E. coli DH10B cells and colonies containing full length hCMV genome were selected. Then the BAC plasmid carrying hCMV genome was extracted and transferred into E. coli SW102 cells that containing a lambda red recombineering system. The hCMV-BAC plasmid can be genetically manipulated within the SW102 cells, and the BAC sequence can be removed by transfecting the BAC containing plasmid into cells which stably expressing Cre recombinase.

FIGS. 2A-2B. Construction of an exemplary tropism-repaired hCMV. FIG. 2A: The unique long (UL) UL131a like sequence in hCMV AD169 strain has been replaced by functional UL131 from Merlin strain. FIG. 2B: Repaired tropism has been demonstrated in U251 human glioma cells.

FIGS. 3A-3B. Construction of exemplary hCMV vectors that can encode and drive synthesis of a lentivirus. FIG. 3A: Two hCMV vectors have been constructed. Each vector expresses a HIV-derived lentivirus vector genome and also contains nucleic acid that can express structural polypeptides for packaging the HIV-derived lentivirus vector genome (e.g., a gag nucleic acid encoding a Gag protein, a pol nucleic acid encoding a DNA polymerase, a rev nucleic acid encoding a transactivator protein, and a nucleic acid encoding a glycoprotein or envelope (Env) protein). One hCMV vector expresses a VSV-G glycoprotein (AD169-BAC R5.3) for packaging the HIV-derived lentivirus vector genome and the other hCMV expresses an amphotropic Murine leukemia virus (MLV) 4070A Env protein (AD169-BAC R5.4) for packaging the HIV-derived lentivirus vector genome. The HIV-derived lentivirus vector genome includes HIV-LTRs, HIV-1 Y, RRE, cPPT/CTS, a transgene (dTomato is used here as a reporter gene) driven by promoter sequence, and an enhancer sequence. FIG. 3B: Reporter gene expression from AD169-BAC R5.4 is demonstrated in MRC-5 human fibroblasts at 9 days post transfection (dpt).

FIG. 4. hCMV vectors can encode and drive synthesis of an infectious lentivirus. hCMV vector (AD169-BAC R5.3 or AD169-BAC R5.4) was transfected into 293T cells. Supernatant was collected and refreshed daily. The collected supernatant was then added to fresh 293T cells. Lentivirus-transduced dTomato GFP-293T red plaques were observed and counted at 4 days post infection.

FIG. 5. Optimization of lentivirus synthesis efficiency for hCMV R5.4. Mutant hCMV R5.4 vectors were constructed by deleting US1-US11, US14-US22, or UL23, and designated as R5.4.1, R5.4.2, and R5.4.4, respectively. The R5.4.1, R5.4.2, and R5.4.4 vectors were transfected into 293T cells. Supernatant was collected at days 5 and 8 post transfection. The collected supernatant was then added to fresh 293T cells. Lentivirus-transduced dTomato GFP 293T red plaques were observed and counted at 4 days post infection.

FIG. 6. Optimization of lentivirus synthesis efficiency for hCMV R5.3 and R5.4. Mutant hCMV R5.4/R5.3 vectors were constructed by deleting US14-US22, US30, UL23, UL83 (pp65) or UL83 (pp71) and designated as R5.4.2/R5.3.2, R5.4.3/R5.3.3, R5.4.4/R5.3.4, R5.4.5/R5.3.5, R5.4.6/R5.3.6, respectively. The mutant vectors were transfected into 293T cells. Supernatant was collected at days 3 and 6 post transfection. The collected supernatant was then added to fresh 293T cells. Lentivirus-transduced dTomato GFP-293T red plaques were observed and counted at 4 days post infection.

FIG. 7. Construction of an exemplary hCMV vector that can encode and drive synthesis of a lentivirus in a controllable manner. The vector expresses an HIV-derived lentivirus vector genome and also contains nucleic acid that can express structural polypeptides for packaging the HIV-derived lentivirus vector genome (e.g., a gag nucleic acid encoding a Gag protein, a pol nucleic acid encoding a DNA polymerase, a rev nucleic acid encoding a transactivator protein, and a nucleic acid encoding an Env protein). Expression of the transactivator protein and a VSV-G Env protein are driven by a drug-controllable promoter such as a tetracycline (tet)-off system (pRSV-tTA-pTight). The HIV-derived lentivirus vector genome includes HIV-LTRs, HIV-1 Y′, RRE, cPPT/CTS, and a transgene (CD19CAR) driven by the lymphocyte specific CD43 promoter sequence. dTomato was used as a reporter gene.

### DETAILED DESCRIPTION

This document relates to methods and materials involved in treating a mammal (e.g., a human) having cancer and/or an infectious disease. For example, this document provides recombinant hCMV vectors that are replication-competent within infected cells, and are designed to infect cells within a mammal to produce hCMV vector-infected cells within the mammal that in turn produce and release a viral vector (e.g., a heterologous viral gene transfer vector such as a lentiviral vector that can infect other cells (e.g., T cells) in vivo and/or can be recognized and internalized by an APC) and can, optionally, drive expression of an exogenous polypeptide (e.g., a therapeutic polypeptide or an antigen receptor such as a CAR) in the infected cells within a mammal (e.g., a human) to induce an immune response within the mammal. In some cases, a viral vector produced by and released from a cell infected by a hCMV vector provided herein can include nucleic acid encoding an exogenous polypeptide (e.g., a therapeutic polypeptide or an antigen receptor such as a CAR) such that the when the viral vector infects a cell (e.g., an immune cell such as a T cell recruited by the hCMV vector-infected cell), the viral vector-infected cell can express the exogenous polypeptide. For example, a recombinant hCMV vector provided herein can be designed such that a viral vector (e.g., a heterologous viral gene transfer vector such as a lentiviral vector) produced by and released from a hCMV vector-infected cell can include nucleic acid encoding a therapeutic polypeptide such that the viral vector can infect a cell (e.g., an immune cell such as a T cell recruited by the hCMV vector-infected cell) and the viral vector-infected cell can express the therapeutic polypeptide. For example, a recombinant hCMV vector provided herein can be designed such that a viral vector (e.g., a heterologous viral gene transfer vector such as a lentiviral vector) produced by and released from a hCMV vector-infected cell can include nucleic acid encoding a CAR such that the viral vector can infect a cell (e.g., an immune cell such as a T cell recruited by the hCMV vector-infected cell) and the viral vector-infected cell can express the CAR resulting in the in vivo generation of a CAR⁺ immune cell (e.g., a CAR⁺ T cell). In some cases, a viral vector produced by and released from a cell infected by a hCMV vector provided herein can be derived from a pathogenic virus such that the viral vector is internalized by an APC. For example, a recombinant hCMV vector provided herein can be designed such that a viral vector (e.g., a heterologous viral gene transfer vector such as a lentiviral vector) produced by and released from a hCMV vector-infected cell can be recognized as a pathogen by an APC such that the viral vector is internalized by the APC and one or more polypeptides from the viral vector are presented as antigens to immune cells (e.g., T cells).

A recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can infect any type of cell. In some cases, a recombinant hCMV vector provided herein can infect fibroblasts, endothelial cells, monocytes, and/or glial cells.

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can be replication competent (e.g., can be replication competent within a hCMV vector-infected cell).

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can be non-pathogenic (e.g., to a mammal being treated as described herein).

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can infect dividing cells (e.g., can infect only dividing cells).

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can infect non-dividing cells (e.g., can infect only non-dividing cells).

A recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can be derived from (e.g., can include genomic elements such as nucleic acids encoding a polypeptide (or fragments thereof)) from any appropriate hCMV. Examples of hCMVs include, without limitation, a hCMV AD169 strain, a hCMV Merlin strain, a hCMV Towne strain, a hCMV TB40 strain, and a hCMV Toledo strain.

A recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include one or more heterologous nucleotide sequences. As used herein a heterologous nucleotide sequence can be any nucleotide sequence that does not naturally occur in that hCMV (e.g., do not naturally occur in that hCMV prior to recombination). Nucleotide sequences that do not naturally occur in the hCMV can be from any appropriate source. In some cases, a nucleotide sequence that does not naturally occur in that hCMV can be from a non-viral organism. In some cases, a nucleotide sequence that does not naturally occur in that hCMV can be from a virus other than a hCMV (e.g., a lentivirus such as a human immunodeficiency virus (HIV), an adeno-associated virus (AAV), a retrovirus, a vesicular stomatitis virus (VSV), or a murine leukemia virus (MLV) such as an amphotropic MLV). In some cases, a nucleotide sequence that does not naturally occur in that hCMV can be from a CMV obtained from a different species. In some cases, a nucleotide sequence that does not naturally occur in that hCMV can be from a different strain of hCMV (e.g., serotypically distinct strains). In some cases, a nucleotide sequence that does not naturally occur in that hCMV can be a synthetic nucleotide sequence.

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include one or more nucleic acids that allow a hCMV vector-infected cell to produce and release a viral vector (e.g., a heterologous viral gene transfer vector such as a lentiviral vector). For example, a recombinant hCMV vector provided herein can include nucleic acids that can encode one or more polypeptides for amplifying and/or packaging a viral vector genome (e.g., a heterologous viral gene transfer vector genome) into infectious vector particles containing the viral vector genome. In some cases, a recombinant hCMV vector can include a viral vector genome (e.g., a viral vector genome derived from a virus other than hCMV) including viral vector packaging elements and can include one or more nucleic acids encoding a viral vector packaging polypeptide (e.g., a polypeptide capable of packaging the viral vector genome to produce a viral vector).

A viral vector genome that can be encoded by a recombinant hCMV vector provided herein can be any appropriate viral vector genome. In some cases, a viral vector genome is not a herpesviral vector genome. In some cases, a viral vector genome is a viral gene transfer vector genome. Examples of viral vector genomes that can be encoded by a recombinant hCMV vector provided herein include, without limitation, lentiviral vector genomes, retroviral vector genomes, AAV vector genomes, picornavirus vector genomes, rhabdovirus vector genomes, and coronavirus vector genomes. Viral vector packaging elements that can be included in a viral vector genome encoded by a recombinant hCMV vector provided herein include, without limitation, long terminal repeats (LTRs) including a 5′ LTR and a 3′ LTR (each of which can include a U3 region, a R region, and a U5 region), a psi (Ψ) element, a Rev response element (RRE), and a central polypurine tract/central termination sequence (cPPT/CTS). Nucleic acid encoding a viral vector packaging polypeptide (e.g., a polypeptide capable of packaging the viral vector genome to produce a viral vector) that can be included in a recombinant hCMV vector provided herein can encode any appropriate viral vector packaging polypeptide. Examples of nucleic acids encoding a viral vector packaging polypeptide include, without limitation, gag nucleic acids (e.g., nucleic acid encoding a group-specific antigen (Gag) protein), pol nucleic acids (e.g., nucleic acid encoding a DNA polymerase), rev nucleic acids (e.g., nucleic acid encoding a transactivator protein), and env nucleic acids (e.g., nucleic acid encoding an envelope protein, with or without a glycoprotein).

In some cases, a viral vector genome (e.g., a viral gene transfer vector genome such as a lentiviral vector genome) that can be encoded by a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) also can include a transgene (e.g., nucleic acid encoding a therapeutic polypeptide or an antigen receptor such as a CAR). In cases where a recombinant hCMV vector provided herein includes a transgene, the transgene can be any appropriate transgene. In some cases, a transgene can be a nucleotide sequence encoding a detectable label. Examples of detectable labels include, without limitation, fluorophores (e.g., green fluorescent protein (GFP), mCherry, yellow fluorescent protein (YFP), cyan fluorescent protein (CFP), and dTomato), enzymes (e.g., luciferase, CRISPR associated protein 9 (Cas9), Cre recombinase, restriction enzymes, convertases, thymidine kinases, and sodium/iodide symporters (NISs)).

In some cases, a transgene that can be included in a viral vector genome (e.g., a viral gene transfer vector genome such as a lentiviral vector genome) that can be encoded by a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include a nucleotide sequence encoding a receptor (e.g., a CAR). A receptor, such as a CAR can target any appropriate antigen (e.g., a cancer antigen). Examples of antigens that can be targeted by a receptor encoded by a transgene in a recombinant hCMV vector provided herein include, without limitation, cluster of differentiation 19 (CD19; associated with B cell lymphomas, acute lymphoblastic leukemia (ALL), and chronic lymphocytic leukemia (CLL)), B-cell maturation antigen (BCMA: associated with multiple myeloma (MM)), alphafetoprotein (AFP; associated with germ cell tumors and/or hepatocellular carcinoma), carcinoembryonic antigen (CEA; associated with bowel cancer, lung cancer, and/or breast cancer), CA-125 (associated with ovarian cancer), mucin 1 (MUC-1; associated with breast cancer), epithelial tumor antigen (ETA; associated with breast cancer), melanoma-associated antigen (MAGE; associated with malignant melanoma), human epidermal growth factor receptor 2 (HER2; associated with breast cancer), biotin (associated with EGFRvIII positive cancer), EGFRvIII (associated with glioma), CD171 (associated with neuroblastoma), and mesothelin (associated with mesothelioma, pancreatic cancer and non-small cell lung cancer).

In some cases, a transgene that can be included in a viral vector genome (e.g., a viral gene transfer vector genome such as a lentiviral vector genome) that can be encoded by a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include a nucleotide sequence encoding a therapeutic polypeptide. Examples of therapeutic polypeptides include, without limitation, immunomodulatory factors such as chemokines and cytokines, antibodies such as antibodies blocking immune checkpoint molecules (e.g., PD-1, PDL-1, and CTLA-4), and CD47 polypeptides.

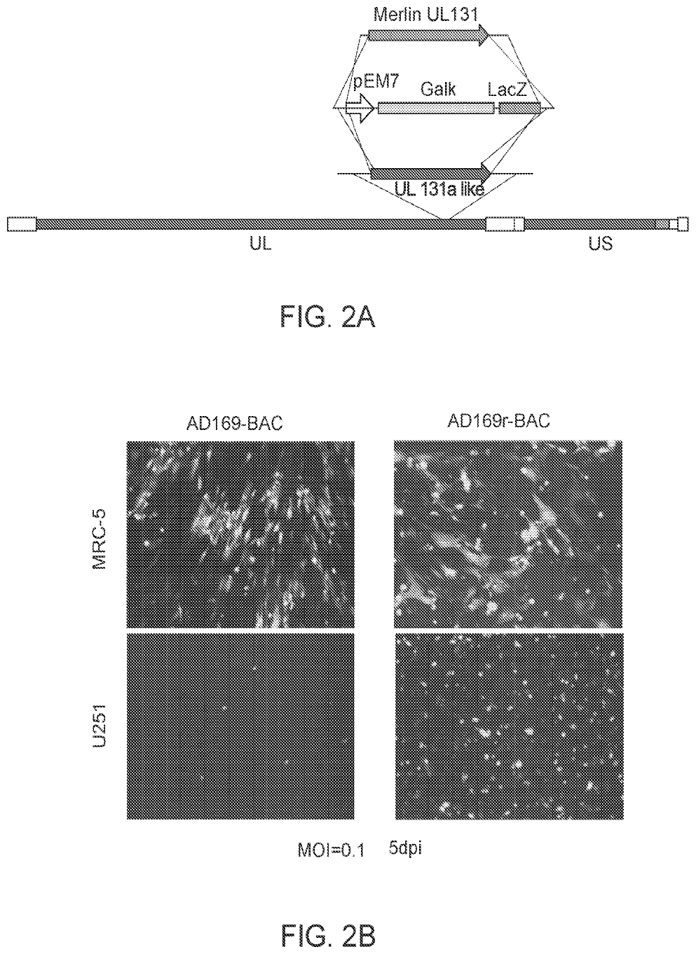

When a transgene that can be included in a viral vector genome (e.g., a viral gene transfer vector genome such as a lentiviral vector genome) that can be encoded by a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) includes a nucleotide sequence encoding CAR, the CAR can be any appropriate CAR. A CAR can include an antigen-binding domain, an optional hinge, a transmembrane domain, and one or more signaling domains. An antigen-binding domain of a CAR that can be expressed by a viral vector (e.g., a heterologous viral gene transfer vector such as a lentiviral vector) produced by and released from a cell infected by a recombinant hCMV vector provided herein that can be administered to a mammal (e.g., a human) as described herein can be any appropriate antigen-binding domain. In some cases, an antigen-binding domain can include an antibody or a fragment thereof that targets an antigen (e.g., a cancer antigen such as a CD19 polypeptide). Examples of antigen-binding domains include, without limitation, an antigen-binding fragment (Fab), a variable region of an antibody heavy (VH) chain, a variable region of a light (VL) chain, a single chain variable fragment (scFv), and domains from growth factors that bind to a cancer cell-specific receptor (e.g., domains from EGF, PDGR, FGF, TGF, or derivatives thereof). In some cases, an antigen-binding domain can target (e.g., can target and bind to) a tumor-specific antigen. For example, a recombinant hCMV vector provided herein can be designed to produce a viral vector (e.g., a heterologous viral gene transfer vector such as a lentiviral vector) that can express a CAR that can bind to a tumor-specific antigen (e.g., an antigen present on cancer cells with minimal, or no, expression on non-cancerous cell types). In some cases, an antigen-binding domain of a CAR can be as described elsewhere (see, e.g., U.S. Patent Application Publication No. 2017/0183418 such as U.S. Patent Application Publication No. 2017/0183418 at paragraph 0015] and the sequence listing; U.S. Patent Application Publication No. 2017/0183413 such as U.S. Patent Application Publication No. 2017/0183413 at paragraph [0049], FIG. 2, Table 9, and the sequence listing; U.S. Patent Application Publication No. 2018/0291079 such as U.S. Patent Application Publication No. 2018/0291079 at paragraphs [0041]-[0045], and Table 4; U.S. Patent Application Publication No. 2020/0289563 such as U.S. Patent Application Publication No. 2020/0289563 at paragraphs [0006]-[0053], [0186]-[0189], and Table 1; and U.S. Patent Application Publication No. 2003/0211097 such as U.S. Patent Application Publication No. 2003/0211097 at paragraphs [0081] and [0211-0215] and the sequence listing.

In some cases, a CAR that can be expressed by a viral vector (e.g., a heterologous viral gene transfer vector such as a lentiviral vector) produced by and released from a cell infected by a recombinant hCMV vector provided herein can include an optional hinge region. In some cases, a hinge region can be located between an antigen-binding domain and a transmembrane domain of a CAR. In some cases, a hinge region can provide a CAR with increased flexibility for the antigen-binding domain. For example, a hinge region can reduce spatial limitations of an antigen-binding domain of a CAR and its target antigen (e.g., to increase binding between an antigen-binding domain of a CAR and its target antigen). Examples of hinge regions that can be used as described herein include, without limitation, a membrane-proximal region from an IgG, a membrane-proximal region from CD8, and a membrane-proximal region from CD28. In some cases, a hinge region of a CAR can be as described elsewhere (see, e.g., U.S. Patent Application Publication No. 2018/0000914 such as U.S. Patent Application Publication No. 2018/0000914 at paragraph [0168], and Table 1; U.S. Patent Application Publication No. 2017/0183418 such as U.S. Patent Application Publication No. 2017/0183418 at paragraphs [0034], [0037], [0040], and Table 2; U.S. Patent Application Publication No. 2017/0183413 such as U.S. Patent Application Publication No. 2017/0183413 at paragraph [0116]; and U.S. Patent Application Publication No. 2017/0145094 such as U.S. Patent Application Publication No. 2017/0145094 at paragraph [0104].

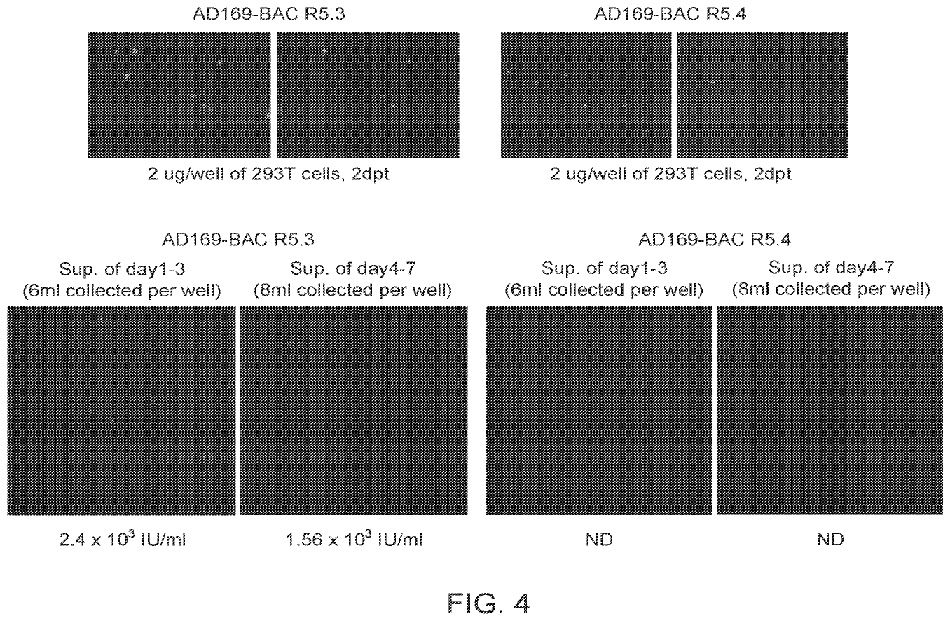

A transmembrane domain of a CAR that can be expressed by a viral vector (e.g., a heterologous viral gene transfer vector such as a lentiviral vector) produced by and released from a cell infected by a recombinant hCMV vector provided herein that can be administered to a mammal (e.g., a human) as described herein can include any appropriate transmembrane domain. A transmembrane domain can be located between an antigen-binding domain and a signaling domain of a CAR and/or located between a hinge and a signaling domain of a CAR. In some cases, a transmembrane domain can provide structural stability for the CAR. For example, a transmembrane domain can include a structure (e.g., a hydrophobic alpha helix structure) that can span a cell membrane and can anchor the CAR to the plasma membrane. Examples of transmembrane domains that can be used as described herein include, without limitation, CD32 transmembrane domains, CD4 transmembrane domains, CD8 (e.g., a CD8a) transmembrane domains, CD28 transmembrane domains, CD16 transmembrane domains, and erythropoietin receptor transmembrane domains. In some cases, a transmembrane domain of a CAR can be as described elsewhere (see, e.g., U.S. Patent Application Publication No. 2016/0120906 such as U.S. Patent Application Publication No. 2016/0120906 at paragraphs [0155], [0161], [0269], FIG. 4, and FIG. 11; U.S. Patent Application Publication No. 2019/0209616 such as U.S. Patent Application Publication No. 2019/0209616 at paragraph [0026]; U.S. Patent Application Publication No. 2018/0000914 such as U.S. Patent Application Publication No. 2018/0000914 at paragraphs [0168]-[0171]; U.S. Patent Application Publication No. 2017/0183418 such as U.S. Patent Application Publication No. 2017/0183418 at paragraphs [0116]-[0118]; U.S. Patent Application Publication No. 2017/0183413 such as U.S. Patent Application Publication No. 2017/0183413 at paragraphs [0116]-[0118]; and U.S. Patent Application Publication No. 2017/0145094 such as U.S. Patent Application Publication No. 2017/0145094 at paragraphs [0104]-[0107].

The one or more signaling domains of a CAR that can be expressed by a viral vector (e.g., a heterologous viral gene transfer vector such as a lentiviral vector) produced by and released from a cell infected by a recombinant hCMV vector provided herein that can be administered to a mammal (e.g., a human) as described herein can include any appropriate signaling domain or combination of signaling domains (e.g., a combination of two, three, or four signaling domains). In some cases, a signaling domain of a CAR can be an intracellular signaling domain normally found within T cells or NK cells. Examples of signaling domains that can be used as described herein include, without limitation, CD2 signaling domains, CD3ζ signaling domains, CD28 signaling domains, Toll-like receptor (TLR) signaling domains (e.g., TLR3 or TLR4 signaling domains), CD27 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, FceR1y intracellular signaling domains, CD278 intracellular signaling domains, CD122 intracellular signaling domains, CD132 intracellular signaling domains, CD70 intracellular signaling domains, cytokine receptor intracellular signaling domains, and CD40 intracellular signaling domains. In some cases, a CAR for use as described herein can be designed to be a first generation CAR having a CD3 intracellular signaling domain. In some cases, a CAR for use as described herein can be designed to be a second generation CAR having a CD28 intracellular signaling domain followed by a CD3ζ intracellular signaling domain. In some cases, a CAR for use as described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, the intracellular signaling domain(s) of a CAR can be as described elsewhere (see, e.g., U.S. Patent Application Publication No. 2018/0000914 such as U.S. Patent Application Publication No. 2018/0000914 at paragraphs [0164]-[0167]; and U.S. Patent Application Publication No. 2017/0183413 such as U.S. Patent Application Publication No. 2017/0183413 at paragraphs [0112]-[0115].

Examples of CARs that can be expressed by a viral vector (e.g., a heterologous viral gene transfer vector such as a lentiviral vector) produced by and released from a cell infected by a recombinant hCMV vector provided herein as described herein include, without limitation, EGFRvIII CARs, GD2 CARS, IL-13RA CARS, CD19 CARS, BCMA CARS, CD138 CARs, NKG2-D CARS, HER2 CARS, CD137 CARs, and B7-H3 CARs.

In addition to a transgene (e.g., nucleic acid encoding a therapeutic polypeptide or an antigen receptor such as a CAR), a viral vector genome that can be encoded by a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) also can contain one or more regulatory elements operably linked to the transgene. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a viral vector genome depends on several factors, including, without limitation, inducibility, targeting, and the level of expression desired. For example, a promoter can be included in a viral vector genome to facilitate transcription of a transgene. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner (e.g., T cell specific promoters, NK cell specific promoters, B cell specific promoters, and NKT cell specific promoters). Examples of promoters that can be used to drive expression of an importin polypeptide in cells include, without limitation, cytomegalovirus/chicken beta-actin (CBA) promoters, cytomegalovirus (CMV) promoters, ubiquitin C (UbC) promoters, EF1a promoters, Rous sarcoma virus long terminal repeat (RSV) promoters, human T-lymphotropic virus type I (HTLV-I) promoters, CD43 promoters, B29 promoters, CD14 promoters, and CD3D promoters. As used herein, “operably linked” refers to positioning of a regulatory element in a viral vector genome relative to a transgene in such a way as to permit or facilitate expression of the encoded polypeptide. For example, a viral vector genome can contain a promoter and transgene. In this case, the promoter is operably linked to the transgene such that it drives expression of the transgene in cells.

In some cases, a viral vector genome that can be encoded by a recombinant hCMV vector provided herein can include a nucleic sequence set forth in SEQ ID NO: 1 (see, e.g., Example 2).

In some cases, a viral vector genome that can be encoded by a recombinant hCMV vector provided herein can be as described elsewhere (see, e.g., U.S. Patent Application Publication No. 2004/014702A1 at, for example, FIG. 2).

In some cases, a genome of a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include a functional UL132 sequence. For example, a genome of a recombinant hCMV vector provided herein can include a UL132 sequence that can encode a functional UL132 polypeptide that can allow the recombinant hCMV vector to be replication-competent (e.g., maintain infectious virus production) within an infected cell within a mammal.

In some cases, a genome of a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include one or more modifications to one or more nucleic acids encoding a polypeptide and/or one or more viral elements of the hCMV genome. The one or more modifications can be any appropriate modification. In some cases, one or more modifications within the hCMV genome can alter tropism of the recombinant hCMV vector. In some cases, one or more modification within the hCMV genome can promote virus latency. In some cases, one or more modifications within the hCMV genome can recruit myeloid cells. In some cases, one or more modifications within the hCMV genome can minimize or inhibit an immune responses against the recombinant hCMV vector. Examples of modifications that can be made to a nucleic acid encoding a polypeptide or to a viral element include, without limitation, deletions, insertions, and substitutions. For example, a recombinant hCMV vector provided herein can include one or more deletions within the hCMV genome. In some cases, a recombinant hCMV vector provided herein can have a deletion (e.g., a full deletion or a partial deletion) of at least one (e.g., one, two, three, four, five, six, seven, eight, nine, ten, eleven, twelve, thirteen, fourteen fifteen, or more) of UL23, US1-US11, US30, US14-22, US30, UL83, UL82, UL138, UL144, UL146, UL147, and miR-UL148D. For example, a recombinant hCMV vector provided herein can have a deletion of at least one of UL23, US1-US11, US30, US14-22, US30, UL83, UL82, UL138, UL144, UL146, UL147, and miR-UL148D such that the recombinant hCMV vector lacks nucleic acid encoding a UL23 polypeptide, nucleic acid encoding a US1-US11 polypeptide, nucleic acid encoding a US30 polypeptide, nucleic acid encoding a US14-22 polypeptide, nucleic acid encoding a US30 polypeptide, nucleic acid encoding a UL83 polypeptide, nucleic acid encoding a UL82 polypeptide, nucleic acid encoding a UL138 polypeptide, nucleic acid encoding a UL144 polypeptide, nucleic acid encoding a UL146, and/or nucleic acid encoding a UL147 polypeptide. For example, a recombinant hCMV vector provided herein can have a deletion (e.g., a full deletion or a partial deletion) of at least one (e.g., one, two, three, four, five, six, seven, eight, nine, ten, eleven, twelve, thirteen, fourteen fifteen, or more) of UL23, US1-US11, US30, US14-22, US30, UL83, UL82, UL138, UL144, UL146, and UL147 such that the recombinant hCMV vector lacks a UL23 polypeptide, a US1-US11 polypeptide, a US30 polypeptide, a US14-22 polypeptide, a US30 polypeptide, a UL83 polypeptide, a UL82 polypeptide, a UL138 polypeptide, a UL144 polypeptide, a UL146, and/or a UL147 polypeptide. In some cases, a recombinant hCMV vector provided herein can include one or more substitutions within the hCMV genome. For example, a recombinant hCMV vector provided herein can have a substitution of a UL131 sequence with a heterologous UL131 sequence (e.g., substitution of a defective AD169 UL131a sequence with a functional UL131 sequence from a hCMV Merlin strain) or can have (e.g., can be engineered to have) a repaired UL131 sequence (e.g., deletion of a single A nucleotide from a defective AD169 UL131a sequence at position 77). For example, a recombinant hCMV vector provided herein can have a UL131 sequence having a nucleotide sequence set forth in SEQ ID NO:2 (see, e.g., Example 3).

In cases where a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) includes one or more modifications to the hCMV genome, the one or more modifications can be effective to increase production of a viral vector (e.g., a heterologous viral gene transfer vector such as a lentiviral vector) from the recombinant hCMV vector (e.g., as compared to a recombinant hCMV vector lacking the mutation(s)). For example, a recombinant hCMV vector having a deletion of UL23, US1-US11, US30, US14-22, US30, UL83, and/or UL82 can have increased production of the viral vector from the recombinant hCMV vector. In some cases, a recombinant hCMV vector having a deletion of UL23, US1-US11, US30, US14-22, US30, UL83, and/or UL82 can produce from about 10³ transduction units (TU) per mL supernatant to about 10⁶ TU/mL supernatant (e.g., from about 10³ TU/mL to about 10⁵ TU/mL, from about 10³ TU/mL to about 10⁴ TU/mL, from about 10⁴ TU/mL to about 10⁶ TU/mL, from about 10⁵ TU/mL to about 10⁶ TU/mL, or from about 10⁴ TU/mL to about 10⁵ TU/mL supernatant). For example, a recombinant hCMV vector having a deletion of UL23, US1-US11, US30, US14-22, US30, UL83, and/or UL82 can produce about 10⁴ TU/mL supernatant

In cases where a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) includes one or more modifications to the hCMV genome, the one or more modifications can be effective to alter the tropism of the recombinant hCMV vector (e.g., as compared to a recombinant hCMV vector lacking the mutation(s)). For example, a recombinant hCMV vector having a UL131 sequence from a hCMV Merlin strain, a hCMV Towne strain, a hCMV TB40 strain, or a hCMV Toledo strain can infect cell types that recombinant hCMV vector lacking the mutation(s) cannot infect. In some cases, a recombinant hCMV vector having a UL131 sequence from a hCMV Merlin strain, a hCMV Towne strain, a hCMV TB40 strain, or a hCMV Toledo strain can infect epithelial cells, endothelial cells, monocytes, and glial cells in addition to fibroblasts.

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include (e.g., can be designed to include) a lentiviral (e.g., HIV) gag nucleic acid, a lentiviral (e.g., HIV) pol nucleic acid, a lentiviral (e.g., HIV) rev nucleic acid, and a VSV-G env nucleic acid, as well as nucleic acid encoding a lentiviral genome including lentiviral (e.g., HIV) LTRs, a lentiviral (e.g., HIV) psi (Ψ) element, a lentiviral (e.g., HIV) RRE, and a lentiviral (e.g., HIV) cPPT/CTS.

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can include (e.g., can be designed to include) a lentiviral (e.g., HIV) gag nucleic acid, a lentiviral (e.g., HIV) pol nucleic acid, a lentiviral (e.g., HIV) rev nucleic acid, and a MLV env nucleic acid, as well as nucleic acid encoding a lentiviral genome including lentiviral (e.g., HIV) LTRs, a lentiviral (e.g., HIV) psi (Ψ) element, a lentiviral (e.g., HIV) RRE, and a lentiviral (e.g., HIV) cPPT/CTS.

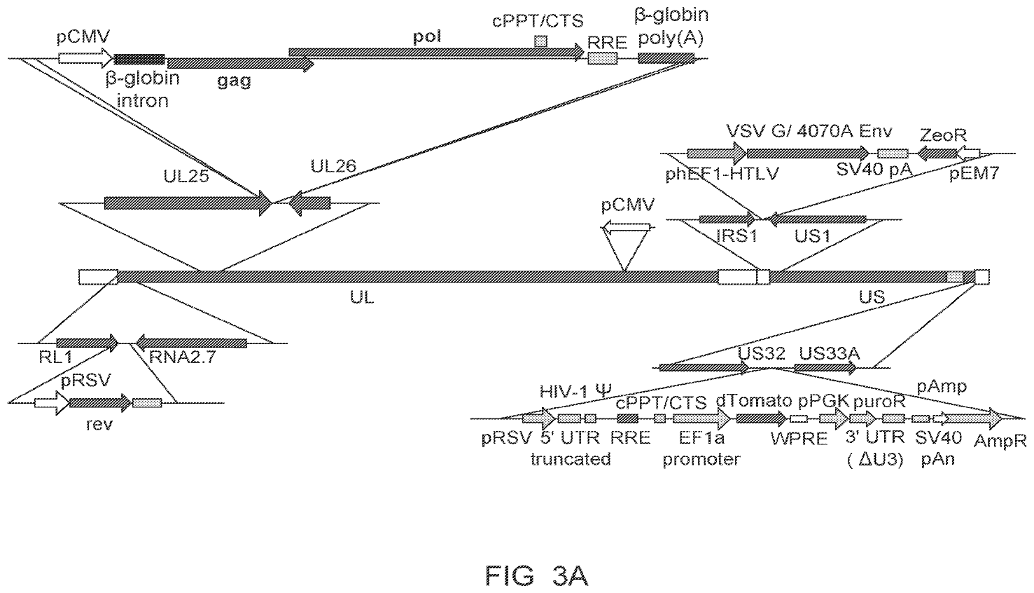

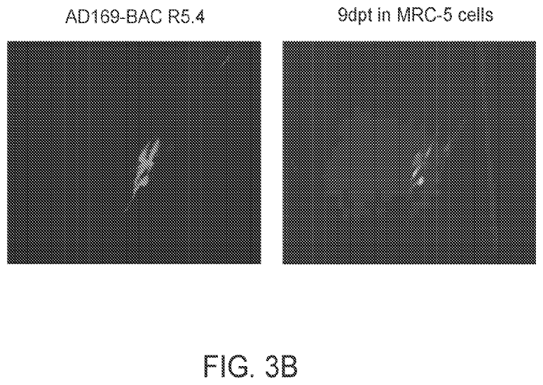

In some cases, a recombinant hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can be as shown in FIG. 3A.

A viral vector produced by and released from a cell infected by a hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can infect any type of cell. In some cases, a viral vector produced by and released from a cell infected by a hCMV vector provided herein can infect an immune cell. Examples of immune cells that can be infected by a viral vector produced by and released from a cell infected by a hCMV vector provided herein include, without limitation, T cells, NK cells, and NKT cells.

In some cases, viral vector produced by and released from a cell infected by a hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can be replication defective (e.g., can be replication defective within a viral vector-infected cell such as an viral vector-infected immune cell).

In some cases, viral vector produced by and released from a cell infected by a hCMV vector provided herein (e.g., a recombinant hCMV vector designed to infect cells within a mammal to produce hCMV vector-infected cells within the mammal that in turn produce and release a viral vector such as a lentiviral vector that can infect other cells such as T cells in vivo and/or can be recognized and internalized by an APC) can integrate into the genome of an infected cell (e.g., can integrate into the genome of a viral vector-infected cell such as an viral vector-infected immune cell).

This document also provides methods and materials for using recombinant hCMV vectors provided herein (e.g., one or more recombinant hCMV vectors designed to infect cells within a mammal to produce hCMV vector-infected cells within the mammal that in turn produce and release a viral vector such as a lentiviral vector that can infect other cells such as T cells in vivo and/or can be recognized and internalized by an APC). In some cases, a recombinant hCMV vector provided herein can used to treat a mammal having, or at risk of having cancer and/or an infectious disease. For example, methods for treating a mammal having cancer and/or an infectious disease can include administering one or more recombinant hCMV vectors provided herein to the mammal. When treating a mammal (e.g., a human) having cancer, one or more recombinant hCMV vectors provided herein can be administered to the mammal to reduce the number of cancer cells in the mammal (e.g., suppress and/or delay tumor growth) and/or to increase survival of the mammal. When treating a mammal (e.g., a human) having an infectious disease, one or more recombinant hCMV vectors provided herein can be administered to the mammal to reduce the number of infected cells in the mammal and/or to reduce the number of or eliminate the pathogens (e.g., pathogens causing the infectious disease) present within the mammal.

Any appropriate mammal (e.g., a mammal having cancer and/or an infectious disease) can be treated as described herein (e.g., by administering one or more recombinant hCMV vectors provided herein). Examples of mammals that can be treated as described herein include, without limitation, humans, non-human primates (e.g., monkeys), dogs, cats, horses, cows, pigs, sheep, mice, and rats. In some cases, a human can be administered one or more recombinant hCMVs provided herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein.

When the methods and materials provided herein are used to treat a mammal (e.g., a human) having cancer, the mammal can have any type of cancer. A cancer to be treated as described herein can be a primary cancer or a metastatic cancer. In some cases, a cancer treated as described herein can include one or more solid tumors. In some cases, a cancer treated as described herein can be a blood cancer. Examples of cancers that can be treated as described herein include, without limitation, brain cancers (e.g., glioblastoma), B cell lymphomas, ALL, CLL, neuroblastomas, breast cancers, and lung cancers.

When the methods and materials provided herein are used to treat a mammal (e.g., a human) having cancer, the methods also can include identifying the mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can be administered or instructed to self-administer one or more recombinant hCMVs described herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein.

When the methods and materials provided herein are used to treat a mammal (e.g., a human) having an infectious disease, the mammal can have, or can be at risk of developing, any type of infectious disease. For example, a mammal having an infectious disease can be administered or instructed to self-administer one or more recombinant hCMVs described herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein to reduce the number of pathogens causing the infectious disease within the mammal. For example, a mammal having an infectious disease can be administered or instructed to self-administer one or more recombinant hCMVs described herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein to reduce or eliminate one or more symptoms of the infectious disease. For example, a mammal at risk of developing an infectious disease can be administered or instructed to self-administer one or more recombinant hCMVs described herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein prior to exposure to a pathogen that can cause an infectious disease to reduce the risk of developing the infectious disease. In some cases, an infectious disease can be caused by a virus. Examples of infectious diseases that can be treated as described herein include, without limitation, viral pneumonia, acquired immune deficiency syndrome (AIDS), and viral hepatitis.

When the methods and materials provided herein are used to treat a mammal (e.g., a human) having an infectious disease, the methods also can include identifying the mammal as having the infectious disease. Examples of methods for identifying a mammal as having an infectious disease include, without limitation, laboratory tests (e.g., laboratory tests of blood samples, urine samples, throat swab samples, stool samples, and/or spinal tap samples) to check the sample for a particular microbe) and/or imaging scans (e.g., X-rays, computerized tomography (CT), and magnetic resonance imaging (MRI)). Once identified as having cancer, a mammal can be administered or instructed to self-administer one or more recombinant hCMVs provided herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein.

One or more recombinant hCMVs provided herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein can be administered by any appropriate route, e.g., intravenous, intramuscular, subcutaneous, oral, intranasal, inhalation, transdermal, and parenteral, to a mammal. In some cases, one or more recombinant hCMVs provided herein or a nucleic acid (e.g., an expression vector) encoding a recombinant hCMV vector provided herein can be administered intravenously to a mammal (e.g., a human).

One or more recombinant hCMVs provided herein (e.g., one or more recombinant hCMVs designed to produce and release a viral vector that can infect cells in vivo and/or can be recognized and internalized by an APC) or a nucleic acid (e.g., an expression vector) encoding a recombinant hCMV vector provided herein can be formulated into a composition (e.g., a pharmaceutical composition) for administration to a mammal (e.g., a mammal having cancer and/or an infectious disease). For example, one or more recombinant hCMVs provided herein or a nucleic acid (e.g., an expression vector) encoding a recombinant hCMV vector provided herein can be formulated into a pharmaceutically acceptable composition for administration to a mammal having cancer and/or an infectious disease. In some cases, one or more recombinant hCMVs provided herein or a nucleic acid (e.g., an expression vector) encoding a recombinant hCMV vector provided herein can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The invention will be further described in the following examples, which do not limit the scope of the invention described in the claims.

### EXAMPLES

### Example 1: Recombinant Human Cytomegalovirus Vector Synthesizing Non-Herpes Viral Particles and Uses Thereof

This Example describes the design of a hCMV vector that can be used for in vivo gene editing of immune cells.

hCMV BAC Reconstruction System

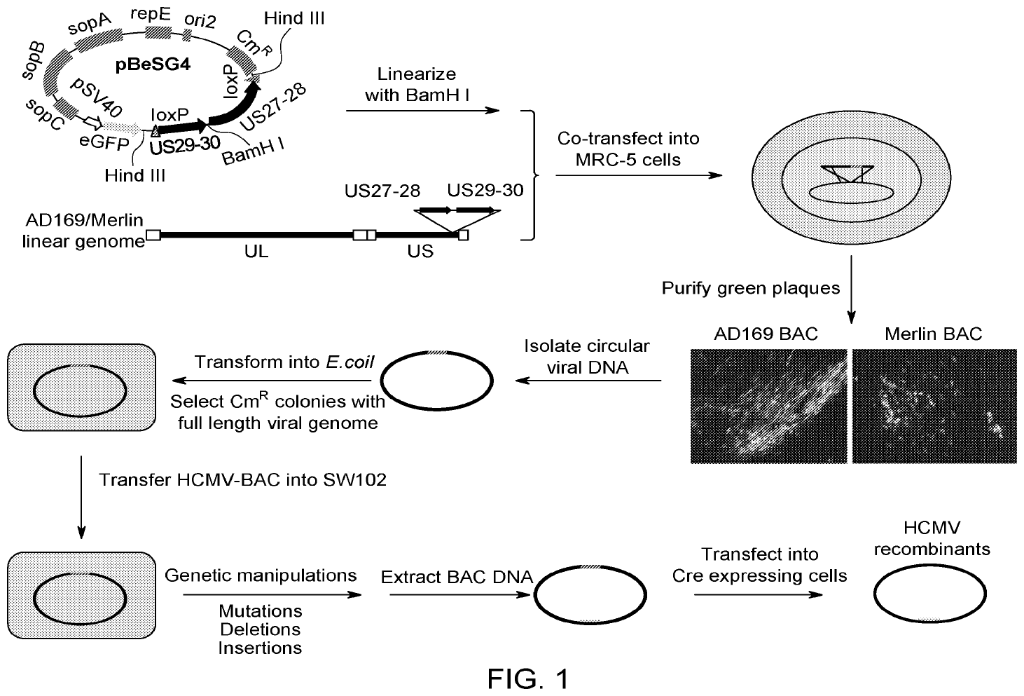

The bacterial artificial chromosome (BAC) technology has been proven to be an invaluable tool for reconstructing herpesviruses, since the DNA genome of herpesvirus is too large to be cloned into single plasmid or cosmid. A BAC vector can hold up to 300 kb DNA sequence, and a hCMV BAC can be created by recombination mediated integration of BAC vector into the circular hCMV DNA. When combined with the lambda red recombineering system, various types of genetic manipulations including insertion, deletion point mutation and fusion can be introduced into the hCMV genome. BAC technology and the lambda red recombineering system were used to construct all the recombinant viruses and vectors (FIG. 1).

Tropism Repaired hCMV hCMV has endothelialtropic (mediated by gH/gL/UL128-UL131) and non-endothelialtropic (mediated by gH/gL/(gO)) pathways to enter into cells. The integrity of those two pathways controls the cell tropism of hCMV. The function of gH/gL/UL128-UL131 complex was repaired to improve cell fusion of hCMV infected cells and endow hCMV the tropism to infect epithelial cells, endothelial cells, and monocytes (FIG. 2), which would improve the infectivity of hCMV vectors.
Construction of hCMV Vector Synthesizing Lentivirus: AD169-BAC Gag-Pol-Rev-VSVG/4070A-LV-EF1a-Dtomato (AD169-BAC R5.3/R5.4)

Based on hCMV AD169-BAC, an HIV derived lentivirus vector genome, HIV helper functions (gag, pol and rev) for packaging the vector genome, and one envelope protein (either VSV-G glycoprotein or amphotropic murine leukemia virus 4070A Env envelope protein), were inserted to different genome sites of AD169-BAC, and two hCMV vectors, AD169-BAC R5.3 (expresses VSVG) and AD169-BAC R5.4 (expresses 4070A) were constructed (FIG. 3). The HIV derived lentivirus gene transfer vector genome inserted consists of HIV-LTRs, HIV-1 Y, RRE, cPPT/CTS, a transgene driven by promoter sequence, and an enhancer sequence.

Efficiency of lentivirus production by the two hCMV vectors was tested in 293T cells, and AD169-BAC R5.3 vector presented a higher lentivirus production (FIG. 4).

Optimization of Lentivirus Synthesis Efficiency for hCMV Vector AD169-BAC R5.4

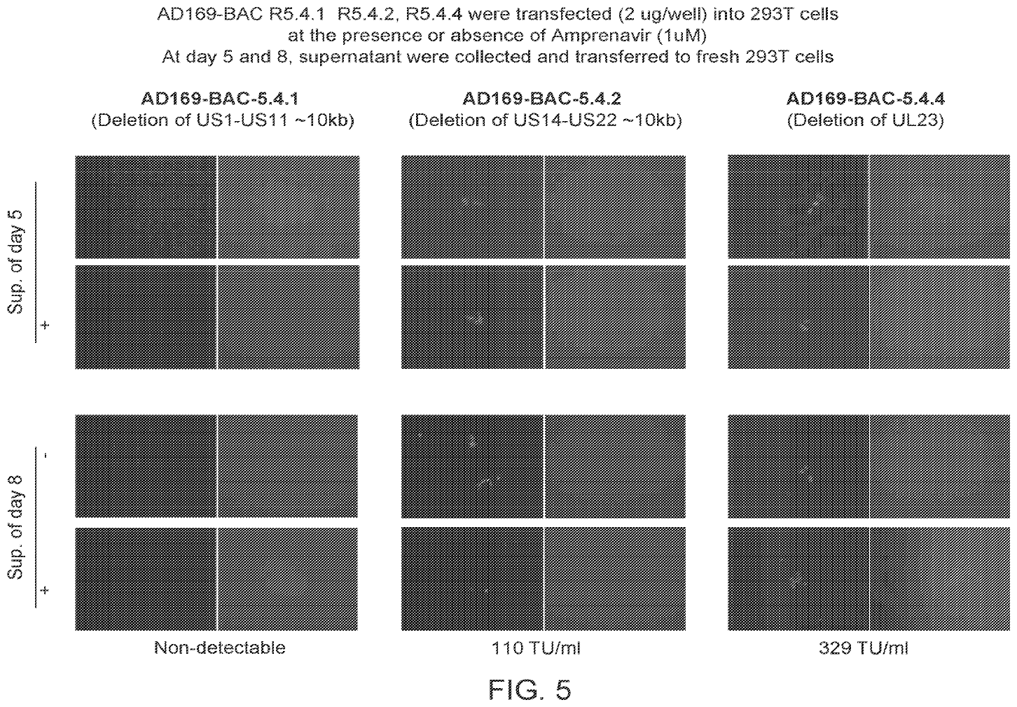

To further optimize the lentivirus synthesis efficiency of the hCMV vectors, three pieces of regions from hCMV genome were deleted from AD169-BAC R5.4 backbone. The deletion of either US14-US22 or UL23 improved the infectious lentivirus production from non-detectable to hundreds of transduction units (TU) per mL supernatant (FIG. 5). These results demonstrate that mutation of hCMV non-essential genes can increase lentivirus production efficiency.

Optimization of Lentivirus Synthesis Efficiency for hCMV Vectors AD169-BAC R5.3 and R5.4

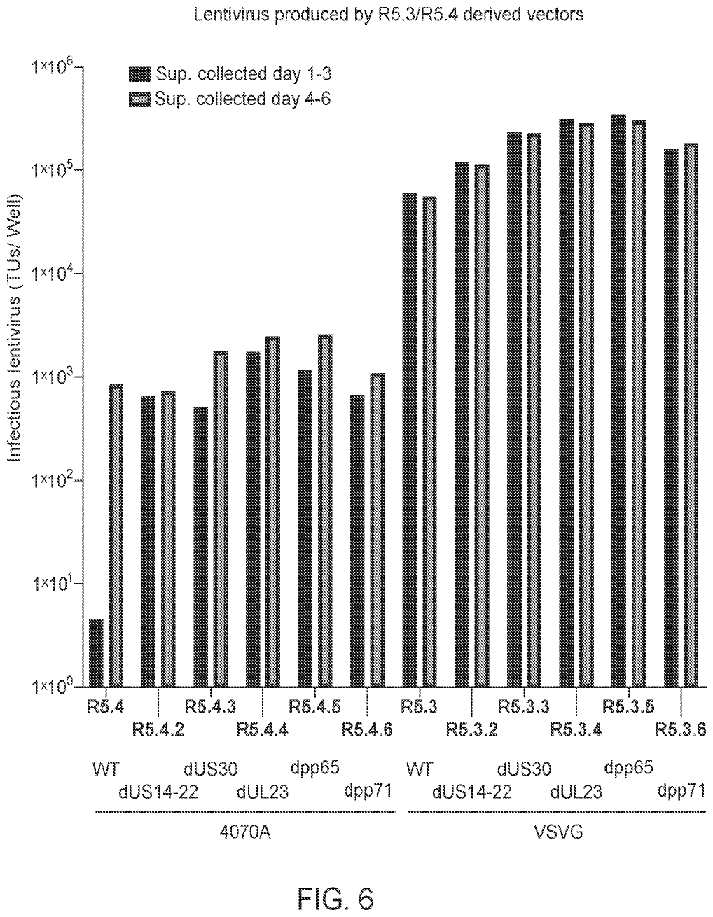

To further optimize the lentivirus synthesis efficiency of the hCMV vectors, five pieces of regions from hCMV genome were deleted from AD169-BAC R5.3 and R5.4 backbone. The deletion of US14-US22, US30, UL23, UL83, or UL82 improved the infectious lentivirus production from non-detectable to 10² to 10⁴ of transduction units (TU) per mL supernatant (FIG. 6). These results demonstrate that mutation of hCMV non-essential genes can further increase lentivirus production efficiency.

### Example 2: Genome Sequence of an Exemplary Lentiviral Gene Transfer Vector

Genome sequence of a LV-EF1a-hCD19CAR-T2A-dTomato lentiviral gene transfer vector that can be encoded by a recombinant hCMV vector provided herein.

SEQ ID NO: 1
GGGTCTCTCTGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGCAGTGGC
GCCCGAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAATTTT
GACTAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTAAAA
CATATAGTATGGGCAAGCAGGGAGCTAGAACGATTCGCAGTTAATCCTGGCCTGTTAGAAAC
ATCAGAAGGCTGTAGACAAATACTGGGACAGCTACAACCATCCCTTCAGACAGGATCAGAAG
AACTTAGATCATTATATAATACAGTAGCAACCCTCTATTGTGTGCATCAAAGGATAGAGATA
AAAGACACCAAGGAAGCTTTAGACAAGATAGAGGAAGAGCAAAACAAAAGTAAGACCACCGC
ACAGCAAGCGGCCGCTGATCTTCAGACCTGGAGGAGGAGATATGAGGGACAATTGGAGAAGT
GAATTATATAAATATAAAGTAGTAAAAATTGAACCATTAGGAGTAGCACCCACCAAGGCAAA
GAGAAGAGTGGTGCAGAGAGAAAAAAGAGCAGTGGGAATAGGAGCTTTGTTCCTTGGGTTCT
TGGGAGCAGCAGGAAGCACTATGGGCGCAGCGTCAATGACGCTGACGGTACAGGCCAGACAA
TTATTGTCTGGTATAGTGCAGCAGCAGAACAATTTGCTGAGGGCTATTGAGGCGCAACAGCA
TCTGTTGCAACTCACAGTCTGGGGCATCAAGCAGCTCCAGGCAAGAATCCTGGCTGTGGAAA
GATACCTAAAGGATCAACAGCTCCTGGGGATTTGGGGTTGCTCTGGAAAACTCATTTGCACC
ACTGCTGTGCCTTGGAATGCTAGTTGGAGTAATAAATCTCTGGAACAGATTTGGAATCACAC
GACCTGGATGGAGTGGGACAGAGAAATTAACAATTACACAAGCTTAATACACTCCTTAATTG
AAGAATCGCAAAACCAGCAAGAAAAGAATGAACAAGAATTATTGGAATTAGATAAATGGGCA
AGTTTGTGGAATTGGTTTAACATAACAAATTGGCTGTGGTATATAAAATTATTCATAATGAT
AGTAGGAGGCTTGGTAGGTTTAAGAATAGTTTTTGCTGTACTTTCTATAGTGAATAGAGTTA
GGCAGGGATATTCACCATTATCGTTTCAGACCCACCTCCCAACCCCGAGGGGACCCGACAGG
CCCGAAGGAATAGAAGAAGAAGGTGGAGAGAGAGACAGAGACAGATCCATTCGATTAGTGAA
CGGATCTCGACGGTATCGCTAGCTTTTAAAAGAAAAGGGGGGATTGGGGGGTACAGTGCAGG
GGAAAGAATAGTAGACATAATAGCAACAGACATACAAACTAAAGAATTACAAAAACAAATTA
CAAAAATTCAAAATTTTACTAGTGATTATCGGATCAACTTTGTATAGAAAAGTTGGGCTCCG
GTGCCCGTCAGTGGGCAGAGCGCACATCGCCCACAGTCCCCGAGAAGTTGGGGGGAGGGGTC
GGCAATTGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGCCGTGA
ACGTTCTTTTTCGCAACGGGTTTGCCGCCAGAACACAGGTAAGTGCCGTGTGTGGTTCCCGC
GGGCCTGGCCTCTTTACGGGTTATGGCCCTTGCGTGCCTTGAATTACTTCCACCTGGCTGCA
GTACGTGATTCTTGATCCCGAGCTTCGGGTTGGAAGTGGGTGGGAGAGTTCGAGGCCTTGCG
CTTAAGGAGCCCCTTCGCCTCGTGCTTGAGTTGAGGCCTGGCCTGGGCGCTGGGGCCGCCGC
GTGCGAATCTGGTGGCACCTTCGCGCCTGTCTCGCTGCTTTCGATAAGTCTCTAGCCATTTA
AAATTTTTGATGACCTGCTGCGACGCTTTTTTTCTGGCAAGATAGTCTTGTAAATGCGGGCC
AAGATCTGCACACTGGTATTTCGGTTTTTGGGGCCGCGGGCGGCGACGGGGCCCGTGCGTCC
CAGCGCACATGTTCGGCGAGGCGGGGCCTGCGAGCGCGGCCACCGAGAATCGGACGGGGGTA
GTCTCAAGCTGGCCGGCCTGCTCTGGTGCCTGGTCTCGCGCCGCCGTGTATCGCCCCGCCCT
GGGCGGCAAGGCTGGCCCGGTCGGCACCAGTTGCGTGAGCGGAAAGATGGCCGCTTCCCGGC
CCTGCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGTACC
GGGCGCCGTCCAGGCACCTCGATTAGTTCTCGAGCTTTTGGAGTACGTCGTCTTTAGGTTGG
GGGGAGGGGTTTTATGCGATGGAGTTTCCCCACACTGAGTGGGTGGAGACTGAAGTTAGGCC
AGCTTGGCACTTGATGTAATTCTCCTTGGAATTTGCCCTTTTTGAGTTTGGATCTTGGTTCA
TTCTCAAGCCTCAGACAGTGGTTCAAAGTTTTTTTCTTCCATTTCAGGTGTCGTGACAAGTT
TGTACAAAAAAGCAGGCTggatccGCCACCatggccttaccagtgaccgccttgctcctgcc
gctggccttgctgctccacgccgccaggccggacatccagatgacacagactacatcctccc
tgtctgcctctctgggagacagagtcaccatcagttgcagggcaagtcaggacattagtaaa
tatttaaattggtatcagcagaaaccagatggaactgttaaactcctgatctaccatacatc
aagattacactcaggagtcccatcaaggttcagtggcagtgggtctggaacagattattctc
tcaccattagcaacctggagcaagaagatattgccacttacttttgccaacagggtaatacg
cttccgtacacgttcggaggggggaccaagctggagatcacaggtggcggtggctcgggcgg
tggtgggtcgggtggcggcggatctgaggtgaaactgcaggagtcaggacctggcctggtgg
cgccctcacagagcctgtccgtcacatgcactgtctcaggggtctcattacccgactatggt
gtaagctggattcgccagcctccacgaaagggtctggagtggctgggagtaatatggggtag
tgaaaccacatactataattcagctctcaaatccagactgaccatcatcaaggacaactcca
agagccaagttttcttaaaaatgaacagtctgcaaactgatgacacagccatttactactgt
gccaaacattattactacggtggtagctatgctatggactactggggccaaggaacctcagt
caccgtctcctcaaccacgacgccagcgccgcgaccaccaacaccggcgcccaccatcgcgt
cgcagcccctgtccctgcgcccagaggcgtgccggccagcggcggggggcgcagtgcacacg
agggggctggacttcgcctgtgatatctacatctgggcgcccttggccgggacttgtggggt
ccttctcctgtcactggttatcaccctttactgcaaacggggcagaaagaaactcctgtata
tattcaaacaaccatttatgagaccagtacaaactactcaagaggaagatggctgtagctgc
cgatttccagaagaagaagaaggaggatgtgaactgagagtgaagttcagcaggagcgcaga
cgcccccgcgtacaagcagggccagaaccagctctataacgagctcaatctaggacgaagag
aggagtacgatgttttggacaagagacgtggccgggaccctgagatggggggaaagccgaga
aggaagaaccctcaggaaggcctgtacaatgaactgcagaaagataagatggcggaggccta
cagtgagattgggatgaaaggcgagcgccggaggggcaaggggcacgatggcctttaccagg
gtctcagtacagccaccaaggacacctacgacgcccttcacatgcaggccctgccccctcgc
GGATCTGGCGAGGGCAGAGGCAGTCTGCTGACATGCGGTGACGTGGAAGAGAATCCCGGCCC
Tcatatgtcagtgagcaagggcgaggaggtcatcaaagagttcatgcgcttcaaggtgcgca
tggagggctccatgaacggccacgagttcgagatcgagggcgagggcgagggccgcccctac
gagggcacccagaccgccaagctgaaggtgaccaagggcggccccctgcccttcgcctggga
catcctgtccccccagttcatgtacggctccaaggcgtacgtgaagcaccccgccgacatcc
ccgattacaagaagctgtccttccccgagggcttcaagtgggagcgcgtgatgaacttcgag
gacggcggtctggtgaccgtgacccaggactcctccctgcaggacggcacgctgatctacaa
ggtgaagatgcgcggcaccaacttcccccccgacggccccgtaatgcagaagaagaccatgg
gctgggaggcctccaccgagcgcctgtacccccgcgacggcgtgctgaagggcgagatccac
caggccctgaagctgaaggacggcggccactacctggtggagttcaagaccatctacatggc
caagaagcccgtgcaactgcccggctactactacgtggacaccaagctggacatcacctccc
acaacgaggactacaccatcgtggaacagtacgagcgctccgagggccgccaccacctgttc
ctgtacggcatggacgagctgtacaagtaatctagaACCCAGCTTTCTTGTACAAAGTGGTG
ATAATCGAATTCCGATAATCAACCTCTGGATTACAAAATTTGTGAAAGATTGACTGGTATTC
TTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTA
TGAGGAGTTGTGGCCCGTTGTCAGGCAACGTGGCGTGGTGTGCACTGTGTTTGCTGACGCAA
CCCCCACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGGGCTCG
GCTGTTGGGCACTGACAATTCCGTGGTGTTGTCGGGGAAGCTGACGTCCTTTCCATGGCTGC
TCGCCTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCATCGGGAATTCCCGC
GGTTCGAATTCTACCTTAATTAAGGTACCTTTAAGACCAATGACTTACAAGGCAGCTGTAGA
TCTTAGCCACTTTTTAAAAGAAAAGGGGGGACTGGAAGGGCTAATTCACTCCCAACGAAGAC
AAGATCTGCTTTTTGCTTGTACTGGGTCTCTCTGGTTAGACCAGATCTGAGCCTGGGAGCTC
TCTGGCTAACTAGGGAACCCACTGCTTAAGCCTCAATAAAGCTTGCCTTGAGTGCTTCAAGT
AGTGTGTGCCCGTCTGTTGTGTGACTCTGGTAACTAGAGATCCCTCAGACCCTTTTAGTCAG
TGTGGAAAATCTCTAGCA

### Example 3: Exemplary AD169 UL131 Sequence

ATGCGGCTGTGTCGGGTGTGGCTGTCTGTTTGTCTGTGCGCCGTGGTGCTGGGTCAGTGCCA
GCGGGAAACCGCGGAAAAAAACGATTATTACCGAGTACCGCATTACTGGGACGCGTGCTCTC
GCGCGCTGCCCGACCAAACCCGTTACAAGTATGTGGAACAGCTCGTGGACCTCACGTTGAAC
TACCACTACGATGCGAGCCACGGCTTGGACAACTTTGACGTGCTCAAGAGGTGAGGGTACGC
GCTAAAGGTGCATGACAACGGGAAGGTAAGGGCGAACGGGTAACGGCTAAGTAACCGCATGG
GGTATGAAATGACGTTTGGAACCTGTGCTTGCAGAATCAACGTGACCGAGGTGTCGTTGCTC
ATCAGCGACTTTAGACGTCAGAACCGTCGCGGCGGCACCAACAAAAGGACCACGTTCAACGC
CGCCGGTTCGCTGGCGCCACACGCCCGGAGCCTCGAGTTCAGCGTGCGGCTCTTTGCCAACT
AG

### Example 4: In Vivo Generation of CAR T Cells

Recombinant hCMV vectors that can produce and release a viral vector (e.g., a heterologous viral gene transfer vector such as a lentiviral vector) that can infect T cells in vivo and can drive expression of a CAR in the infected T cells within a mammal (e.g., a human) to generate CAR⁺ T cells within the mammal are engineered by replacing the promoter with a T cell-specific promoter. For example, the nucleic acid encoding a dTomato polypeptide as shown in FIG. 3A is replaced with a transgene encoding a CD19 CAR, and the EF1a promoter as shown in FIG. 3A is replaced with a T cell-specific promoter (e.g., a CD3D promoter).

### Example 5: Construction of an Inducible hCMV Vector Capable of Synthesizing Lentivirus

Based on hCMV AD169-BAC, an hCMV vector that can encode and drive synthesis of a lentivirus in a drug-controllable manner was constructed. The following steps were used to generate an hCMV vector that can be induced to synthesize a lentivirus.

Nucleic acid that contains pRSV-tTA-pTight-rev was inserted between hCMV IRS1 and US1 via homologous recombination.

Nucleic acid that contains pTight-VSV-F-SV40 pA-ZeoR-pEM7 was inserted before the pRSV-tTA-pTight-rev cassette in the hCMV vector.

Nucleic acid that contains pCMV-gag-pol-β-globin polyA was inserted between hCMV UL25 and UL26.

Nucleic acid that contains pRSV-truncated 5′UTR (HIV)-HIV-1 Y-RRE-cPPT/CTS-pCD43-CD19CAR-dTomato-WPRE-5′UTR (ΔU3)-SV40 pA-pAmp-AmpR was inserted between hCMV US32 and US33A.

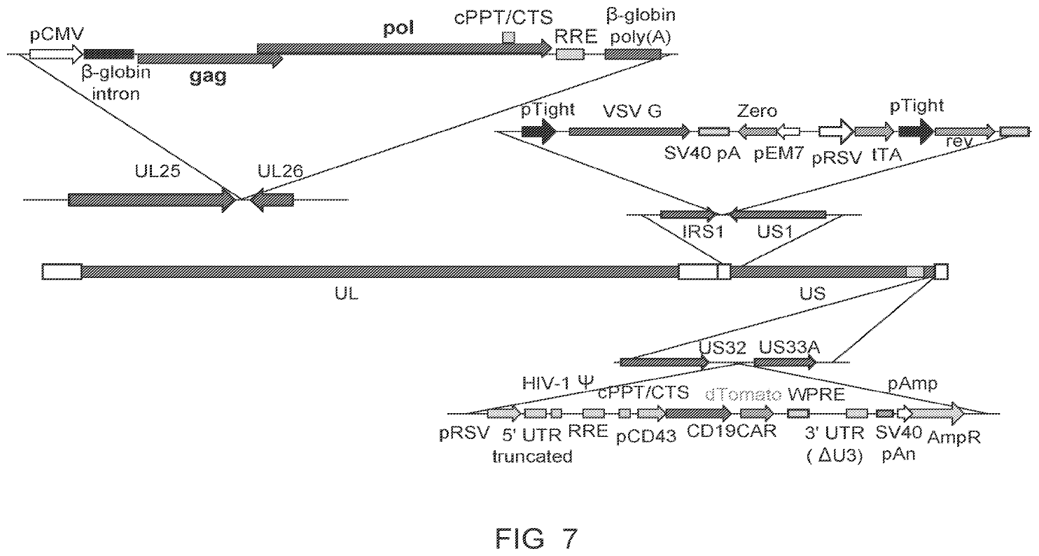

A schematic demonstrating the construction of an hCMV vector that can encode and drive synthesis of a lentivirus in a drug-controllable manner is shown in FIG. 7.

### Example 6: Generation of Inducible hCMV Vector Capable of Synthesizing Lentivirus

High titers of recombinant hCMV vectors that can produce and release a viral vector (e.g., a heterologous viral gene transfer vector such as a lentiviral vector) are produced in vitro. Fibroblast cells (e.g., human fibroblast cells such as MRC-5 or WI-38 cells) are infected with recombinant hCMV vectors that can produce and release a viral vector (e.g., a heterologous viral gene transfer vector such as a lentiviral vector) in the absence of a particular drug (e.g., in the absence of tetracycline or doxycycline), and are administered that particular drug (e.g., tetracycline or doxycycline) to suppress synthesis of the viral vector. For example, fibroblast cells (e.g., human fibroblast cells such as MRC-5 or WI-38 cells) are infected with the hCMV vector as shown in FIG. 7 and are administered tetracycline to suppress synthesis of the viral vector. The recombinant hCMV vectors are replicated by the infected cells generating high titers of the recombinant hCMVs. In some cases, the recombinant hCMV vectors are purified from the cell culture.

### Example 7: In Vivo Generation of CAR T Cells

Recombinant hCMV vectors that can produce and release a viral vector (e.g., a heterologous viral gene transfer vector such as a lentiviral vector) in the absence of a particular drug (e.g., in the absence of tetracycline or doxycycline) are administered to a mammal and infect cells within the mammal. For example, the hCMV vector as shown in FIG. 7 is administered to a mammal. hCMV vector-infected cells produce and release a viral vector (e.g., a heterologous viral gene transfer vector such as a lentiviral vector) that can infect T cells in vivo and can express a CAR to generate CAR⁺ T cells within the mammal.

### Example 8: Treating Cancer

A human identified as having a CD19-positive cancer is administered one or more recombinant hCMV vectors designed to produce and release a viral vector (e.g., a heterologous viral gene transfer vector such as a lentiviral vector) that can express a CD19 CAR and can infect cells in vivo. The recombinant hCMV vector(s) can infect cells within the human such that viral vectors produced by and released from the recombinant hCMV vector(s) can infect T cells within the human and can express a CD19 CAR to generate CD19-CAR T cells within the human. The in vivo generated CD19-CAR T cells can target and destroy CD19-positive cancer cells within the human.

### Example 9: Treating AIDS

A human identified as having AIDS is administered one or more recombinant hCMV vectors designed to produce and release a HIV-derived lentiviral vector. The recombinant hCMV vector(s) can infect cells within the human such that HIV-derived lentiviral vectors produced by and released from the recombinant hCMV vector(s) are internalized by APCs in vivo such that one or more polypeptides from the HIV-derived lentiviral vector are presented as antigens to T cells (e.g., naïve T cells) within the human to induce an immune response against HIV particles present in the human. The immune response against HIV particles present in the human reduces or eliminates one or more symptoms of AIDS.

### Example 10: Preventing AIDS

A human that is identified as being HIV-positive or as being at risk of being exposed to HIV is administered one or more recombinant hCMV vectors designed to produce and release a HIV-derived lentiviral vector. The recombinant hCMV vector(s) can infect cells within the human such that HIV-derived lentiviral vectors produced by and released from the recombinant hCMV vector(s) are internalized by APCs in vivo such that one or more polypeptides from the HIV-derived lentiviral vector are presented as antigens to T cells (e.g., naïve T cells) within the human to prime the T cells. In the presence of HIV particles, the primed T cells induce an immune response against HIV particles present in the human. The immune response against HIV particles present in the human prevents the development of AIDS.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A recombinant human cytomegalovirus (hCMV) vector capable of infecting a cell of a mammal, wherein said hCMV vector comprises heterologous nucleic acid encoding (i) a heterologous viral gene transfer vector genome and (ii) one or more helper polypeptides for amplifying and packaging said heterologous viral gene transfer vector genome into infectious vector particles, wherein said cell infected with said hCMV vector produces and releases said infectious vector particles comprising said gene transfer vector genome.

2. The hCMV vector of claim 1, wherein said hCMV vector is replication-competent in said cell.

3. The hCMV vector of claim 1, wherein said hCMV vector lacks nucleic acid encoding a UL138 polypeptide, a UL144 polypeptide, a UL146 polypeptide, a UL147 polypeptide, or a miR-UL148D.

4. The hCMV vector of claim 1, wherein said hCMV vector lacks a UL138 polypeptide, a UL144 polypeptide, a UL146 polypeptide, a UL147 polypeptide, or a miR-UL148D.

5. (canceled)

6. The hCMV vector of claim 1, wherein said hCMV vector lacks nucleic acid encoding a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide.

7. The hCMV vector of claim 1, wherein said hCMV vector lacks a UL23 polypeptide, a US1 polypeptide, a US2 polypeptide, a US3 polypeptide, a US4 polypeptide, a US5 polypeptide, a US6 polypeptide, a US7 polypeptide, a US8 polypeptide, a US9 polypeptide, a US10 polypeptide, a US11 polypeptide, a US14 polypeptide, a US15 polypeptide, a US16 polypeptide, a US17 polypeptide, a US18 polypeptide, a US19 polypeptide, a US20 polypeptide, a US21 polypeptide, a US22 polypeptide, a US30 polypeptide, a UL82 polypeptide, or a UL83 polypeptide.

8. The hCMV vector of claim 1, wherein said cell is a fibroblast, an epithelial cell, an endothelial cell, a monocyte, or a glial cell.

9. The hCMV vector of claim 1, wherein said viral gene transfer vector genome is a lentiviral vector genome, a retroviral vector genome, an adeno-associated virus (AAV) vector genome, a picornavirus vector genome, a rhabdovirus vector genome, or a coronavirus vector genome.

10. The hCMV vector of claim 1, wherein said viral gene transfer vector genome comprises a nucleic acid sequence encoding a therapeutic polypeptide or an antigen receptor polypeptide, wherein said infectious vector particles comprising said gene transfer vector genome can infect an immune cell within said mammal, and wherein the infected immune cell directs expression of said therapeutic polypeptide or said antigen receptor polypeptide.

11. The hCMV vector of claim 10, wherein said viral gene transfer vector genome comprises a nucleic acid sequence encoding said therapeutic polypeptide.

12. (canceled)

13. The hCMV vector of claim 10, wherein said viral gene transfer vector genome comprises a nucleic acid sequence encoding said antigen receptor polypeptide.

14-15. (canceled)

16. The hCMV vector of claim 10, wherein said immune cell is a T cell, a natural killer (NK) cell, or a natural killer T (NKT) cell.

17. The hCMV vector of claim 10, wherein said viral gene transfer vector is replication-defective in said infected immune cell.

18. The hCMV vector of claim 1, wherein said viral gene transfer vector genome comprises one or more packaging elements selected from the group consisting of a 5′ long terminal repeat (LTR), a 3′ LTR, a psi (Ψ) element, a Rev response element (RRE), and a central polypurine tract/central termination sequence (cPPT/CTS).

19. The hCMV vector of claim 1, wherein said nucleic acid encoding one or more helper polypeptides comprises nucleic acid encoding an envelope polypeptide selected from the group consisting of a vesicular stomatitis virus G (VSV G) polypeptide and a murine leukemia virus (MLV) 4070A polypeptide.

20. The hCMV vector of claim 19, wherein said released viral gene transfer vector comprises said envelope polypeptide.

21. A method for treating a mammal having cancer, wherein said method comprises administering a recombinant hCMV vector to said mammal, wherein said hCMV vector is capable of infecting a cell of said mammal, wherein said hCMV comprises heterologous nucleic acid encoding (i) a heterologous viral gene transfer vector genome comprising a nucleic acid sequence encoding an antigen receptor polypeptide targeting a cancer antigen of said cancer and (ii) one or more helper polypeptides for amplifying and packaging said heterologous viral gene transfer vector genome into infectious vector particles, wherein said cell infected with said hCMV produces and releases said infectious vector particles comprising said gene transfer vector genome, and wherein said released infectious vector particles are capable of infecting an immune cell within said mammal and directing expression of said antigen receptor polypeptide by said infected immune cell, and wherein said infected immune cell expressing said antigen receptor reduces the number of cancer cells within said mammal.

22. The method of claim 21, wherein said mammal is a human.

23-35. (canceled)

36. A method for treating a mammal having an infectious disease, wherein said method comprises administering a recombinant hCMV vector to said mammal, wherein said hCMV vector is capable of infecting a cell of said mammal, wherein said hCMV vector comprises heterologous nucleic acid encoding (i) a heterologous viral gene transfer vector genome derived from a virus causing the infectious disease and (ii) one or more helper polypeptides for amplifying and packaging said heterologous viral gene transfer vector genome into infectious vector particles, wherein said cell infected with said hCMV vector produces and releases said infectious vector particles comprising said gene transfer vector genome, and wherein said infectious vector particles are recognized by an antigen presenting cell (APC) within said mammal such that said APC presents an antigen from said viral gene transfer vector to a T cell within said mammal, and wherein said T cell recognizes and destroys the virus causing the infectious disease within said mammal.

37. The method of claim 36, wherein said mammal is a human.

38-52. (canceled)


SEQUENCE LISTING (2 sequences)

SEQ ID NO: 1 - DNA, 6156 bp, synthetic construct
  source 1..6156: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctggatccgccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggcgagggcagaggcagtctgctgacatgcggtgacgtggaagagaatcccggccctcatatgtcagtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaatctagaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccttaattaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 498 bp, synthetic construct
  source 1..498: mol_type = other DNA
atgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagaggtgagggtacgcgctaaaggtgcatgacaacgggaaggtaagggcgaacgggtaacggctaagtaaccgcatggggtatgaaatgacgtttggaacctgtgcttgcagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactag